(19) United States
(12) Patent Application Publication
(10) Pub. No.: US 2010/0040727 A1
(43) Pub. Date: Feb. 18, 2010

(54) Method for Improving Uniformity of Content in Edible Film Manufacturing

(75) Inventors: Garry L. Myers, Kingsport, IN (US); Madhusudan Hariharan, Munster, IN (US); Pradeep Sanghvi, Schererville, IN (US); Alex Szopinski, Chesterton, IN (US); Patrick Dailey, Merrillville, IN (US)

Correspondence Address: HOFFMANN & BARON, LLP, 6900 JERICHO TURNPIKE, SYOSSET, NY, 11791, US

(73) Assignee: MONOSOL RX, LLC, Portage, IN (US)

(21) Appl. No.: 12/193,079

(22) Filed: Aug. 18, 2008

(51) Int. Cl. A61K 9/70; A23P 1/10

(52) U.S. Cl. 426/2; 426/517; 426/658

(57) ABSTRACT

The present invention relates to a method of making a film composition with an improved uniformity of active composition. The invention includes a method including the steps of forming a flowable film composition comprising an edible water-soluble polymer, a polar solvent and a particulate active suspended therein, where the ratio of solids to liquids is at least about 95 to about 5 and where the composition comprises a mixture having compositional uniformity per unit volume; directing the flowable film composition to a transfer trough, the transfer trough including a first rotating roller and a movable transfer substrate upon which to form the film; and maintaining uniformity of the flowable film composition during the residence time in the trough by maintaining the rate of composition entering the transfer trough and the level of the flowable film composition in the transfer trough at a substantially constant level. Methods of administering the active are also contemplated.

### FIELD OF THE INVENTION

The invention relates to rapidly dissolving films and methods of their preparation. The films may also contain an active ingredient that is evenly distributed throughout the film. The uniform distribution is achieved by controlling one or more parameters, and particularly maintaining a certain ratio of solids to liquids in the film-forming solution, maintaining a substantially constant ingress and egress of the film solution in the transfer trough, and controlling the deposition of the film solution into the transfer trough.

### BACKGROUND OF THE RELATED TECHNOLOGY

Active ingredients, such as drugs or pharmaceuticals, may be prepared in a tablet form to allow for accurate and consistent dosing. However, this form of preparing and dispensing medications has many disadvantages including that a large proportion of adjuvants that must be added to obtain a size able to be handled, that a larger medication form requires additional storage space, and that dispensing includes counting the tablets which has a tendency for inaccuracy. In addition, many persons, estimated to be as much as 28% of the population; have difficulty swallowing tablets. While tablets may be broken into smaller pieces or even crushed as a means of overcoming swallowing difficulties, this is not a suitable solution for many tablet or pill forms. For example, crushing or destroying the tablet or pill form to facilitate ingestion, alone or in admixture with food, may also destroy the controlled release properties.

As an alternative to tablets and pills, films may be used to carry active ingredients such as drugs, pharmaceuticals, and the like. However, historically films and the process of making drug delivery systems therefrom have suffered from a number of unfavorable characteristics that have not allowed them to be used in practice.

Films that incorporate a pharmaceutically active ingredient are disclosed in expired U.S. Pat. No. 4,136,145 to Fuchs, et al. (“Fuchs”). These films may be formed into a sheet, dried and then cut into individual doses. The Fuchs disclosure alleges the fabrication of a uniform film, which includes the combination of water-soluble polymers, surfactants, flavors, sweeteners, plasticizers and drugs. These allegedly flexible films are disclosed as being useful for oral, topical or enteral use. Examples of specific uses disclosed by Fuchs include application of the films to mucosal membrane areas of the body, including the mouth, rectal vaginal, nasal and ear areas.

Examination of films made in accordance with the process disclosed in Fuchs, however, reveals that such films suffer from the aggregation or conglomeration of particles, i.e., self-aggregation, making them inherently non-uniform. This result can be attributed to Fuchs' process parameters, which although not disclosed may include such factors as using a low trough, a single stream feed, unsieved particles and the like to form such non-uniformity. It has been found that the creation of such films by traditional methods results in films that show a “hammock effect.” This is shown where the ends of the film have a greater amount of active than the middle, leaving the middle portions relatively devoid of the active.

When large dosages are involved, a small change in the dimensions of the film would lead to a large difference in the amount of active per film. If such films were to include low dosages of active, it is possible that portions of the film may be substantially devoid of any active. Since sheets of film are usually cut into unit doses, certain doses, particularly those falling in the middle of the sheet, may therefore be devoid of or contain an insufficient amount of active for the recommended treatment. Failure to achieve a high degree of accuracy with respect to the amount of active ingredient in the cut film can be harmful to the patient. For this reason, dosage forms formed by processes such as Fuchs, would not likely meet the stringent standards of governmental or regulatory agencies, such as the U.S. Federal Drug Administration (“FDA”), relating to the variation of active in dosage forms. Currently, as required by various world regulatory authorities, dosage forms may not vary more than 10% in the amount of active present. When applied to dosage units based on films, this virtually mandates that uniformity in the film be present.

The problems of self-aggregation leading to non-uniformity of a film were addressed in U.S. Pat. No. 4,849,246 to Schmidt (“Schmidt”). Schmidt attempted to solve the uniformity problem by forming a multi-layered film. However, his process is a multi-step process that adds expense and complexity and is not practical for commercial use.

Other U.S. patents directly addressed the problems of particle self-aggregation and non-uniformity inherent in conventional film forming techniques. In one attempt to overcome non-uniformity, U.S. Pat. No. 5,629,003 to Horstmann et al. and U.S. Pat. No. 5,948,430 to Zerbe et al. incorporated additional ingredients, i.e. gel formers and polyhydric alcohols respectively, to increase the viscosity of the film prior to drying in an effort to reduce aggregation of the components in the film. These methods have the disadvantage of requiring additional components, which translates to additional cost and manufacturing steps. Such processes also run the risk of exposing the active, i.e., a drug, or vitamin C, or other components to prolonged exposure to moisture and elevated temperatures, which may render it ineffective or even harmful.

The benefits of preparing a film that is substantially uniform in composition, without adding additional components are numerous. In addition to the obvious cost-savings through the minimal materials, there is a greater percentage of films that are pharmaceutically acceptable. Thus, less film will be deemed “unacceptable”, and there will be less waste. Further, with a more uniform distribution of active, there can be provided a lesser amount of active provided to the mixture. When one has a non-uniform distribution of active, there must be more active added to the mixture to compensate for those portions that are non-uniform.

Therefore, there is a need for methods and compositions for film products, which use a minimal number of materials or components, and which provide an improved uniform heterogeneity throughout the volume of the sheet of film (also known as the “web” of film).

### SUMMARY OF THE INVENTION

In one embodiment of the present invention, there is provided a method of making a film product including the steps of: forming a flowable film composition including an edible water-soluble polymer, a polar solvent and a particulate active suspended therein, where the ratio of solids to liquids is at least about 75 to 99% solids to about 25 to 1% liquids, and where the composition includes a mixture having compositional uniformity per unit volume; controllably directing the flowable film composition to a transfer trough, the transfer trough including a first rotating roller and a movable transfer substrate upon which to form the film; and maintaining uniformity of the flowable film composition during the residence time in the trough by maintaining the rate of composition entering the transfer trough and the level of the flowable film composition in the transfer trough at a substantially constant level.

In another embodiment of the present invention, there is provided a method of orally administering an active to an individual including the steps of preparing a film by the steps of forming a flowable film composition including an edible water-soluble polymer, a polar solvent and a particulate active suspended therein, where the ratio of solids to liquids is at least about 95 to about 5 and where the composition includes a mixture having compositional uniformity per unit volume; controllably directing the flowable film composition to a transfer trough, the transfer trough including a first rotating roller and a movable transfer substrate upon which to form the film; and maintaining uniformity of the flowable film composition during the residence time in the trough by maintaining the rate of composition entering the transfer trough and the level of the flowable film composition in the transfer trough at a substantially constant level; and introducing the film to the oral cavity of an individual.

In another embodiment of the present invention, there is provided a method of introducing an active component to liquid including the steps of preparing a film by the steps of forming a flowable film composition including an edible water-soluble polymer, a polar solvent and a particulate active suspended therein, where the ratio of solids to liquids is at least about 95 to about 5 and where the composition includes a mixture having compositional uniformity per unit volume; controllably directing the flowable film composition to a transfer trough, the transfer trough including a first rotating roller and a movable transfer substrate upon which to form the film; and maintaining uniformity of the flowable film composition during the residence time in the trough by maintaining the rate of composition entering the transfer trough and the level of the flowable film composition in the transfer trough at a substantially constant level; placing the film into a liquid; and allowing the film to dissolve.

### BRIEF DESCRIPTION OF THE DRAWINGS

FIG. 1 is a schematic drawing of the film coating apparatus of the present invention.

FIG. 2 is a close-up drawing of the film suspension trough region of the present invention.

FIG. 3 is a graphical depiction of the concentration of the active ingredient at various points along the film web using a control experiment.

FIG. 4 is a graphical depiction of the concentration of the active ingredient at various points along the film web using a doctor roll in the ON position.

FIG. 5 is a graphical depiction of the concentration of the active ingredient at various points along the film web using sieved particles with a doctor roll in the ON position.

FIG. 6 is a graphical depiction of the concentration of the active ingredient at various points along the film web using high level in the trough with a doctor roll in the ON position.

FIG. 7 is a graphical depiction of the concentration of the active ingredient at various points along the film web using a higher coat weight with a doctor roll in the ON position.

FIG. 8 is a graphical depiction of the concentration of the active ingredient at various points along the film web using a lower concentration of solids in the suspension.

FIG. 9 is a graphical depiction of the concentration of the active ingredient at various points along the film web using a higher concentration of solids in the suspension.

FIG. 10 is a graphical depiction of the concentration of the active ingredient at various points along the film web using a control experiment.

FIG. 11 is a graphical depiction of the concentration of the active ingredient at various points along the film web using a doctor roll at of 30% of maximum sneed.

FIG. 12 is a graphical depiction of the concentration of the active ingredient at various points along the film web using a doctor roll at 60% of maximum speed.

FIG. 13 is a graphical depiction of the concentration of the active ingredient at various points along the film web using a doctor roll at 100% of maximnum speed.

FIG. 14 is a graphical depiction of the concentration of the active ingredient at various points along the film web achieved by moving the suspension entry point to various sides of trough.

FIG. 15 is a graphical depiction of the concentration of the active ingredient at various points along the film web using a four-stream manifold.

FIG. 16 is a graphical depiction of the concentration of the active ingredient at various points along the film web achieved by ladling the suspension into trough.

FIG. 17 is a graphical depiction of the concentration of the active ingredient at various points along the film web using a mylar substrate.

FIG. 18 is a graphical depiction of the concentration of the active ingredient at various points along the film web using sieved particles, a high trough level, and a manifold to split the entry stream into four separate streams.

### DETAILED DESCRIPTION OF THE INVENTION

For the purposes of the present invention the term “non-self-aggregating uniform heterogeneity” refers to the ability of the films of the present invention, which are formed from one or more components in addition to a polar solvent, to provide a substantially reduced occurrence of, i.e., little or no, aggregation or conglomeration of components within the film as is normally experienced when films are formed by conventional methods such as the use of non-uniform particles and a single stream feeding manifold. The term heterogeneity, as used in the present invention, includes films that will incorporate a single component, such as a polymer, as well as combinations of components, such as a polymer and an active agent. Uniform heterogeneity includes the substantial absence of aggregates or conglomerates as is common in conventional mixing and heat drying methods used to form films. Preferably the films of the present invention have a uniform distribution of particles, a uniform height, weight, and density.

Furthermore, the films of the present invention have a substantially uniform thickness, which is also not provided by the use of conventional drying methods used for drying water-based polymer systems. The absence of a uniform thickness detrimentally affects uniformity of component distribution throughout the area of a given film. The films made by the present invention reduce the so-called “hammock effect” seen in traditional film manufacturing.

The film products of the present invention are produced by a combination of a properly selected polymer and a polar solvent, optionally including an active ingredient as well as other fillers known in the art. These films provide a non-self-aggregating uniform heterogeneity of the components within them by utilizing a selected casting, deposition or extrusion method, and a controlled drying process. Examples of controlled drying processes include, but are not limited to, the use of the apparatus disclosed in U.S. Pat. No. 4,631,837 to Magoon (“Magoon”), herein incorporated by reference, as well as other drying methods as set forth in the present applicants' co-pending application publication numbers 2005/0184427 and 2007/0069416, which are additionally incorporated herein by reference.

The products and processes of the present invention rely on the interaction among various steps of the production of the films in order to provide films that substantially reduce the self-aggregation of the components within the films. Specifically, these steps include the particular method used to form the film, including using a particular ratio of solids to liquids in the film composition. Additionally, the use of particles that are substantially uniform in thickness may give uniformity. For example, particles that have been sieved to remove large particle size fraction greater than 70 mesh and fines smaller than 140 mesh (approximately 100 to 200 micrometers) have been seen to provide a more uniform variability in film. Using particles with a substantially uniform size is helpful, as when there is a varying degree of size the larger particles not only tend to migrate to the sides, but they may also be more difficult to fit through the film-forming gap in the machinery. For example, particles that have a −60/+70 particle size show an average assay value of 628.50%, while those particles that have a −140/+200 particle size show an assay value of 53.333%. Larger particles have a tendency to coat less. The migration of particles to the sides contributes to the previously discussed “hammock effect”.

In addition to the use of uniformly-sized particles, maintaining the rate of composition entering the transfer trough and maintaining the trough level at a certain height can help maintain uniformity. Keeping the level of the trough and the rate of composition entering the trough substantially constant help reduce the disturbance in the trough. Specifically, when the suspension level in the trough is at a higher level, a significant reduction in variability across the web of the film is seen.

Additionally, the means to introduce the suspension of materials into the trough can have a significant effect on the uniformity of the film. Controllably directing the flowable film composition to a transfer trough may additional help reduce the disturbance in the trough. Traditional means to feed the transfer trough have used a single-stream manifold to feed the film solution into the coating trough, generally centered in the middle of the trough. Such placement has a tendency to ripple the solution in the trough, creating agitation and thus displacing the particles in the solution. However, by using a manifold, which splits the single stream into multiple streams, using a cascading stream, using an oscillating stream, or ladling the composition into the trough, one can reduce the level of disturbance in the trough, creating a more uniform film. Preferably, the stream is split into about three to five streams; however, any number of multiple streams can be used in the present invention. By keeping the disturbance in the trough low, there are fewer tendencies of the particles to shift to the sides of the trough and give a less uniform film.

Desirably, there is less than about a 10% change in the particle content per unit volume throughout the transfer trough. More desirably, there is less than 5% change, and most preferably there is less than 1% change in the particle content per unit volume throughout the transfer trough.

Other traditional film-forming methods may be used in conjunction with the methods of the present invention, including the use of a doctor roll, which is a device which marginally or softly touches the surface of the film and controllably disposes the particles onto the film surface, to level the suspension in the trough. While the use of a doctor roll is optional, the preferred method is to use a doctor roll in the on position while manufacturing the film.

Moreover, the films of the present invention may contain particles that are sensitive to temperature, such as flavors, which may be volatile, or drugs, which may have a low degradation temperature. In such cases, the drying temperature may be decreased while increasing the drying time to adequately dry the uniform films of the present invention. Furthermore, bottom drying also tends to result in a lower internal film temperature as compared to top drying. In bottom drying, the evaporating vapors more readily carry heat away from the film as compared to top drying which lowers the internal film temperature. Such lower internal film temperatures often result in decreased drug degradation and decreased loss of certain volatiles, such as flavors.

Furthermore, particles or particulates may be added to the film-forming composition or matrix after the composition or matrix is cast into a film. For example, particles may be added to the film prior to the drying of the film. Particles may be controllably metered to the film and disposed onto the film through any suitable techniques. Such suitable techniques include the use of a doctor roll as described above, the use of an additional roller to place the particles on the film surface, spraying the particles onto the film surface, and the like. The particles may be placed on either or both of the opposed film surfaces, i.e., the top and/or bottom film surfaces. Desirably, the particles are securably disposed onto the film, such as being embedded into the film. Moreover, such particles are desirably not fully encased or fully embedded into the film, but remain exposed to the surface of the film, such as in the case where the particles are partially embedded or partially encased.

The particles may be any useful organoleptic agent, cosmetic agent, pharmaceutical agent, or combinations thereof. Desirably, the pharmaceutical agent is a taste-masked or a controlled-release pharmaceutical agent. Useful organoleptic agents include flavors and sweeteners. Useful cosmetic agents include breath freshening or decongestant agents, such as menthol, including menthol crystals.

Monitoring and control of the thickness of the film also contributes to the production of a uniform film by providing a film of uniform thickness. The thickness of the film may be monitored with gauges such as Beta Gauges. A gauge may be coupled to another gauge at the end of the drying apparatus, i.e. drying oven or tunnel, to communicate through feedback loops to control and adjust the opening in the coating apparatus, resulting in control of uniform film thickness.

The film products are generally formed by combining a properly selected polymer and polar solvent, as well as any active ingredient or filler as desired. The particles are sieved to achieve substantial uniformity prior to formation. Desirably, the solvent content of the combination is at least about 30% by weight of the total combination. The suspension formed by this combination is split into multiple streams by the use of a manifold to be fed into a trough, preferably a trough with a high level. The material is then formed into a film, desirably by roll coating, and then dried, desirably by a rapid and controlled drying process to maintain the uniformity of the film, more specifically, a non-self-aggregating uniform heterogeneity. The resulting film will desirably contain less than about 10% by weight solvent, more desirably less than about 8% by weight solvent, even more desirably less than about 6% by weight solvent and most desirably less than about 2%. The solvent may be water, a polar organic solvent including, but not limited to, ethanol, isopropanol, acetone, methylene chloride, or any combination thereof.

The ratio of the solid particles to the liquid materials should be kept high so as to maintain uniformity in the film. If the ratio of solid to liquid materials is too low, uniformity will be affected. In situations where the solid portion is too small as compared to the liquid portion, there is a vast amount of space in the solution for the solid particles to move freely, and results in a solution with a lack of uniformity. In contrast, when the ratio of solid particles to liquid materials is high, the particles have less space to move freely, and the particles have a tendency to self-regulate. With other dosage forms, such as tablets and capsules, the volumetric ratio of the piece is set. However, with wet films as described herein, the weight is not set, due to volatiles such as drying steps and mixing with a solvent. Thus, maintaining a high ratio of solid particles to liquid materials is preferable in keeping a uniform solution. Preferably, the film will have a ratio of solid to liquid materials of from about 99 to 1 to about 75 to 25. Most preferably, the film will have a ratio of solid to liquid materials of at least 98 to 2.

Consideration of the above discussed parameters, such as but not limited to using substantially uniformly sized particles, using a manifold to split the suspension into multiple streams, and a higher trough level, also impact material selection for the different components of the present invention. Furthermore, as described above, such consideration with proper material selection provides the compositions of the present invention, including a pharmaceutical and/or cosmetic dosage form or film product, having no more than a 10%, variance of a pharmaceutical and/or cosmetic active per unit area. In other words, the uniformity of the present invention is determined by the presence of no more than a 10% by weight of pharmaceutical and/or cosmetic variance throughout the matrix. Desirably, the variance is less than 5% by weight, less than 2% by weight, less than 1% by weight, or less tharn 0.5% by weight.

### Film-Forming Polymers

The polymer may be water soluble, water swellable, water insoluble, or a combination of one or more either water soluble, water swellable or water insoluble polymers. The polymer may include cellulose or a cellulose derivative. Specific examples of useful water soluble polymers include, but are not limited to, pullulan, hydroxypropylmethyl cellulose hydroxyethyl cellulose, hydroxypropyl cellulose, polyvinyl pyrrolidone, carboxymethyl cellulose, polyvinyl alcohol, sodium aginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy)propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/507 believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers to provide a desired viscosity of the mixture prior to drying. For example, if the active or other components are not soluble in the selected solvent, a polymer that will provide a greater viscosity is desired to assist in maintaining uniformity. On the other hand, if the components are soluble in the solvent, a polymer that provides a lower viscosity may be preferred.

The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof.

It has also been observed that certain polymers which when used alone would ordinarily require a plasticizer to achieve a flexible film, can be combined without a plasticizer and yet achieve flexible films. For example, HPMC and HPC when used in combination provide a flexible, strong film with the appropriate plasticity and elasticity for manufacturing and storage. No additional plasticizer or polyalcohol is needed for flexibility.

### Controlled Release Films

The term “controlled release” is intended to mean the release of active at a pre-selected or desired rate. This rate will vary depending upon the application. Desirable rates include fast or immediate release profiles as well as delayed, sustained or sequential release. Combinations of release patterns, such as initial spiked release followed by lower levels of sustained release of active are contemplated. Pulsed drug releases are also contemplated.

The polymers that are chosen for the films of the present invention may also be chosen to allow for controlled disintegration of the active. This may be achieved by providing a substantially water insoluble film that incorporates an active that will be released from the film over time. This may be accomplished by incorporating a variety of different soluble or insoluble polymers and may also include biodegradable polymers in combination. Alternatively, coated controlled release active particles may be incorporated into a readily soluble film matrix to achieve the controlled release property of the active inside the digestive system upon consumption.

Films that provide a controlled release of the active are particularly useful for buccal, gingival, sublingual and vaginal applications. The films of the present invention are particularly useful where mucosal membranes or mucosal fluid is present due to their ability to readily wet and adhere to these areas.

The convenience of administering a single dose of a medication which releases active ingredients in a controlled fashion over an extended period of time as opposed to the administration of a number of single doses at regular intervals has long been recognized in the pharmaceutical arts. The advantage to the patient and clinician in having consistent and uniform blood levels of medication over an extended period of time are likewise recognized. The advantages of a variety of sustained release dosage forms are well known. However, the preparation of a film that provides the controlled release of an active has advantages in addition to those well-known for controlled release tablets. For example, thin films are difficult to inadvertently aspirate and provide an increased patient compliance because they need not be swallowed like a tablet. Moreover, certain embodiments of the inventive films are designed to adhere to the buccal cavity and tongue, where they controllably dissolve. Furthermore, thin films may not be crushed in the manner of controlled release tablets which is a problem leading to abuse of drugs such as Oxycontin.

The actives employed in the present invention may be incorporated into the film compositions of the present invention in a controlled release form. For example, particles of drug may be coated with polymers such as ethyl cellulose or polymethacrylate, commercially available under brand names such as Aquacoat ECD and Eudragit E-100, respectively. Solutions of drug may also be absorbed on such polymer materials and incorporated into the inventive film compositions. Other components such as fats and waxes, as well as sweeteners and/or flavors may also be employed in such controlled release compositions. The actives may be taste-masked prior to incorporation into the film composition.

### Actives

When an active is introduced to the film, the amount of active per unit area is determined by the uniform distribution off the film. For example, when the films are cut into individual dosage forms, the amount of the active in the dosage form can be known with a great deal of accuracy. This is achieved because the amount of the active in a given area is substantially identical to the amount of active in an area of the same dimensions in another part of the film. The accuracy in dosage is particularly advantageous when the active is a medicament, i.e. a drug.

The active components that may be incorporated into the films of the present invention include, without limitation pharmaceutical and cosmetic actives, drugs, medicaments, antigens or allergens such as ragweed pollen, spores, microorganisms, seeds, mouthwash components, flavors, fragrances, enzymes, preservatives, sweetening agents, colorants, spices, vitamins and combinations thereof.

A wide variety of medicaments, bioactive active substances and pharmaceutical compositions may be included in the dosage forms of the present invention. Examples of useful drugs include ace-inhibitors, antianginal drugs, anti-arrhythmias, anti-asthimatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammation substances, coronary dilators, cerebral dilators, peripheral vasodilators, psycho-tropics, stimulants, anti-hypertensive drugs, vasoconstrictors, migraine treatments, antibiotics, tranquilizers anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythiopoietic drugs, anti-asthmatics, cough suppressants, mucolytics, DNA and genetic modifying drugs, and combinations thereof.

Examples of medicating active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine.

Other preferred drugs for other preferred active ingredients for use in the present invention include anti-diarrheals such as immodium AD, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™) nabumetone (available as Relafen®), and Clemastine (available as Tavist®); anti-emetics such as granisetron hydrochloride (available as Kytril®) and nabilone (available as Cesamet™); bronchodilators such as Bentolin®, albuterol sulfate (available as Proventil®); anti-depressants such as fluoxetine hydrochloride (available as Prozac®), sertrailne hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®).

Erectile dysfunction therapies include, but are not limited to, drugs for facilitating blood flow to the penis, and for effecting autonomic nervous activities, such as increasing parasympathetic (cholinergic) and decreasing sympathetic (adrenersic) activities. Useful non-limiting drugs include sildenafils, such as Viagra®, tadalafils, such as Cialis®, vardenafils, apomorphines, such as Uprima®, yohimbine hydrochlorides such as Aphrodyne®, and alprostadils such as Caverject®.

The popular H₂-antagonists which are contemplated for use in the present invention include cimietidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilyslate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents employed in the present invention may include allergens or antigens, such as, but not limited to, plant pollens from grasses, trees, or ragweed; animal danders, which are tiny scales shed from the skin and hair of cats and other furred animals, insects, such as house dust mites, bees, and wasps; and drugs, such as penicillin.

An anti-oxidant may also be added to the film to prevent the degradation of an active, especially where the active is photosensitive.

Cosmetic active agents may include breath freshening compounds like menthol, other flavors or fragrances, especially those used for oral hygiene, as well as actives used in dental and oral cleansing such as quaternary ammonium bases. The effect of flavors may be enhanced using flavor enhancers like tartanic acid, citric acid, vanillin, or the like.

Also color additives can be used in preparing the films. Such color additives include food, drug and cosmetic colors (FD&C), drag and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cinnamon, cocoa, and citrus oils such as lemon, orange, grape, lime and grapefruit and fruit essences including apple, pear, peach, grape, strawberry, raspberry, cherry, plum, pineapple, apricot or other fruit flavors.

The films containing flavorings may be added to provide a hot or cold flavored drink or soup. These flavorings include, without limitation, tea and soup flavorings such as beef and chicken.

Other useful flavorings include aldehydes and esters such as benzaldehyde (cherry, almond), citral i.e., alphacitral (lemon, lime), neral, i.e., beta-citual (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof, saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; isomalt; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

When the active is combined with the polymer in the solvent, the type of matrix that is formed depends on the solubilities of the active and the polymer. If the active and/or polymer are soluble in the selected solvent, this may form a solution. However, if the components are not soluble, the matrix may be classified as an emulsion, a colloid, or a suspension.

### Dosages

The film products of the present invention are capable of accommodating a wide range of amounts of the active ingredient. The films are capable of providing an accurate dosage amount (determined by the size of the film and concentration of the active in the original polymer/water combination) regardless of whether the required dosage is high or extremely low. Therefore, depending on the type of active or pharmaceutical composition that is incorporated into the film, the active amount may be as high as about 300 mg, desirably up to about 150 mg or as low as the microgram range, or any amount therebetween.

The film products and methods of the present invention are well suited for high potency, low dosage drugs. This is accomplished trough the high degree of uniformity or the films. Therefore, low dosage drugs, particularly more potent racemic mixtures of actives are desirable.

### Anti-Foaming and De-Foaming Compositions

Anti-foaming and/or de-foaming components may also be used with the films of the present invention. These components aid in the removal of air, such as entrapped air, from the film-forming compositions. As described above, such entrapped air may lead to non-uniform films. Simethicone is one particularly useful anti-foaming and/or de-foaming agent. The present invention, however, is not so limited and other anti-foam and/or de-foaming agents may suitable be used.

Simethicone is generally used in the medical field as a treatment for gas or colic in babies. Simethicone is a mixture of fully methylated linear siloxane polymers containing repeating units of polydimethylsiloxane which is stabilized with trimethylsiloxy end-blocking unites, and silicon dioxide. It usually contains 90.5-99% polyethylsiloxane and 4-7% silicon dioxide. The mixture is a gray, translucent, viscous fluid which is insoluble in water.

When dispersed in water, simethicone will spread across the surface, forming a thin film of low surface tension. In this way, simethicone reduces the surface tension of bubbles air located in the solution, such as foam bubbles, causing their collapse. The function of simethicone mimics the dual action of oil and alcohol in water. For example, in an oily solution any trapped air bubbles will ascend to the surface and dissipate more quickly and easily, because an oily liquid has a lighter density compared to a water solution. On the other hand, an alcohol/water mixture is known to lower water density as well as lower the water's surface tension. So, any air bubbles trapped inside this mixture solution will also be easily dissipated. Simethicone solution provides both of these advantages. It lowers the surface energy of any air bubbles that trapped inside the aqueous solution, as well as lowering the surface tension of the aqueous solution. As the result of this unique functionality, simethicone has an excellent anti-foaming property that can be used for physiological processes (anti-gas in stomach) as well as any for external processes that require the removal of air bubbles from a product.

In order to prevent the formation of air bubbles in the films of the present invention, the mixing step can be performed under vacuum. However, as soon as the mixing step is completed, and the film solution is returned to the normal atmosphere condition, air will be re-introduced into or contacted with the mixture. In many cases, tiny air bubbles will be again trapped inside this polymeric viscous solution. The incorporation of simethicone into the film-forming composition either substantially reduces or eliminates the formation of air bubbles.

Simethicone may be added to the film-forming mixture as an anti-foaming agent in an amount from about 0.01 weight percent to about 5.0 weight percent, more desirably from about 0.05 weight percent to about 2.5 weight percent, and most desirably from about 0.1 weight percent to about 1.0 weight percent.

### Optional Components

A variety of other components and fillers may also be added to the films of the present invention. These may include, without limitation, surfactants; plasticizers which assist in compatibilizing the components within the mixture; polyalcohols; anti-foaming agents, such as silicone-containing compounds, which promote a smoother film surface by releasing oxygen from the film; and thermo-setting gels such as pectin, carageenan, and gelatin, which help in maintaining the dispersion of components.

The variety of additives that can be incorporated into the inventive compositions may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quarternized if desired; and other similar polymers.

Such extenders may optionally be added in any desired amount desirably within the range of up to about 80%, desirably about 3% to 50% and more desirably within the range of 3% to 20% based on the weight of all components.

Further additives may be inorganic fillers, such as the oxides of magnesium aluminum, silicon, titanium, etc. desirably in a concentration range of about 0.02% to about 3% by weight and desirably about 0.02% to about 1% based on the weight of all components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosucciniate, triethyl citrate, tributyl citrate, and the like, added in concentrations ranging from about 0.5% to about 30%, and desirably ranging from about 0.5% to about 20% based on the weight of the polymer.

There may further be added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂—, C₁₄—, C₁₆—, C₁₈—, C₂₀— and C₂₂— fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂—, C₁₄—, C₁₆—, C₁₈—, C₂₀— and C₂₂— fatty acids.

The total amounts used of the fats, mono-, di-glycerides and/or lecithins are up to about 5% and preferably within the range of about 0.5% to about 2% by weight of the total composition.

It is further useful to add silicon dioxide, calcium silicate, or titanium dioxide in a concentration of about 0.02% to about 1% by weight of the total composition. These compounds act as texturizing agents.

These additives are to be used in amounts sufficient to achieve their intended purpose. Generally, the combination of certain of these additives will alter the overall release profile of the active ingredient and can be used to modify, i.e. impede or accelerate the release.

Lecithin is one surface active agent for use in the present invention. Lecithin can be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL, which is commercially available from BASF, are also useful. Carbowax™ is yet another modifier which is very useful in the present invention. Tweens™ or combinations of surface active agents may be used to achieve the desired hydrophilic-lipophilic balance (“HLB”). The present invention, however, does not require the use of a surfactant and films or film-forming compositions of the present invention may be essentially flee of a surfactant while still providing the desirable uniformity features of the present invention.

As additional modifiers which enhance the procedure and product of the present invention are identified, Applicants intend to include all such additional modifiers within the scope of the invention claimed herein.

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

### Forming the Film

Although the films of the present invention preferably are described herein according to certain parameters, it will be understood that these may be altered in various ways without substantially affecting the resulting end product. First, the particles to be used in the film are separated, preferably through the use of a sieve, to remove those particles that are too large or small. Preferably, the particles should be in the range of about 100 micrometers to about 200 micrometers in diameter, and more preferably should be more uniform in size, such as between 140 and 160 micrometers. The particles can include polymers, actives, and any other additional materials to be included in the film. After the materials have been sieved to remove those particles that are non-uniform, the desired components are combined to form a multi-component suspension, including the polymer, a solvent, preferably water, and actives or other components as desired. The film composition should have a high ratio of solids to liquids, particularly a ratio of at least about 75 to about 25, and more preferably at least about 95 to about 5, and most preferably about 99 to about 1. The high solid to liquid ratio will aid in maintaining uniformity throughout the film sheet.

The matrix, including the film-forming polymer and polar solvent in addition to any additives and the active ingredient, may be formed in a number of steps. For example, the ingredients may all be added together or a premix may be prepared. The advantage of a pre-mix is that all ingredients except for the active may be combined in advance, with the active added just prior to formation of the film. This is especially important for actives that may degrade with prolonged exposure to water, air or another polar solvent.

In this particular method of forming the matrix, the pre-mix or master batch, which includes the film-forming polymer, polar solvent, and any other additives except for the pharmaceutical active, is added to the master batch feed tank. Then a pre-determined amount of the master batch is controllably fed via a first metering pump and control valve to either or both of the first and second mixers. The required amount of the drug may then be added to the desired mixer through an opening in each of the mixers. After the drug has been blended with the master batch pre-mix for a sufficient time to provide a uniform matrix, a specific amount of the uniform matrix is then ready to be formed into a film.

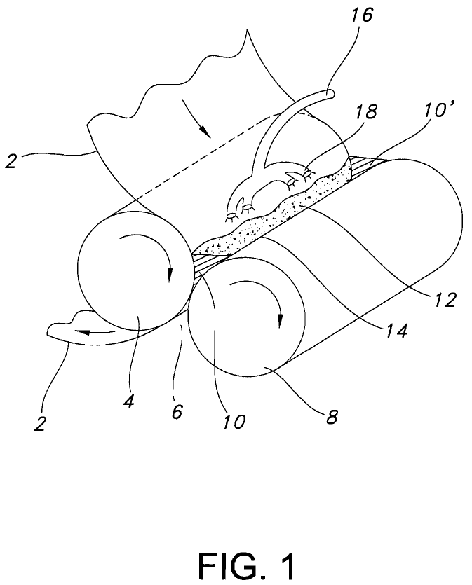

Referring to FIG. 1, the mixture 12 is then fed into a transfer trough 14, which is formed by the close proximity of two rollers, commonly referred to as a drive roll (or applicator roll) 4 and a doctor roll 8. The drive roll 4 and doctor roll 8 form the V-shaped transfer trough 14. The sides of the transfer trough 14 are defined by barriers 10 and 10′, preferably made of plastic. A feeding manifold 16 is used to feed the suspension 12 into the transfer trough 14. A coating substrate 2 is fed along the side of the drive roll 4, where it comes into contact with the suspension 12 in the transfer trough 14. As the substrate 2 travels through the transfer trough 14 an amount of fuel suspension 12 is coated onto the substrate 2. Preferably, the doctor roll 8 spins in the same direction as the drive roll 4, helping to coat the substrate 2. The rate upon which the suspension 12 exits the transfer trough 14 as coated onto the substrate 2 is preferably kept substantially constant.

In accordance with the preferred embodiment described herein, the suspension 12 is controllably directed into the transfer trough 14. The suspension 12 can be controllably directed into the transfer trough 14 by any means desired. FIG. 1 depicts one embodiment of the present invention, which incorporates the use of a split-stream manifold 16, directing multiple streams of suspension 12 into the transfer trough 14. The manifold 16 splits the feed stream into multiple streams 18, most preferably into about three to five streams. Other feeding manifolds 16 are contemplated, such as a “cascading” manifold, which feeds the suspension into the transfer trough 14 across the whole length of the transfer trough 14 or the use of an oscillating manifold, which evenly dispenses the suspension 12 into the transfer trough 14. The use of the controlled feeding of the suspension 12 helps to maintain the uniformity of the suspension 12 during the residence time in the transfer trough 14 by reducing the disruption of the suspension 12 while in the transfer trough 14. Similar to the rate upon which the suspension 12 exits the transfer trough 14, the rate into which the suspension 12 is fed into the transfer trough 14 is preferably kept substantially constant. The maintenance of a constant ingress and egress of the suspension 12 aids in maintaining uniformity of the composition 12 in the transfer trough 14, which in turn helps maintain uniformity of the film sheet. In some embodiments, a user may manually ladle or scoop the suspension 12 into the trough 14. Optionally an agitator bar may be used in the transfer trough 14, helping keep the particles from settling in one place in the transfer trough 14.

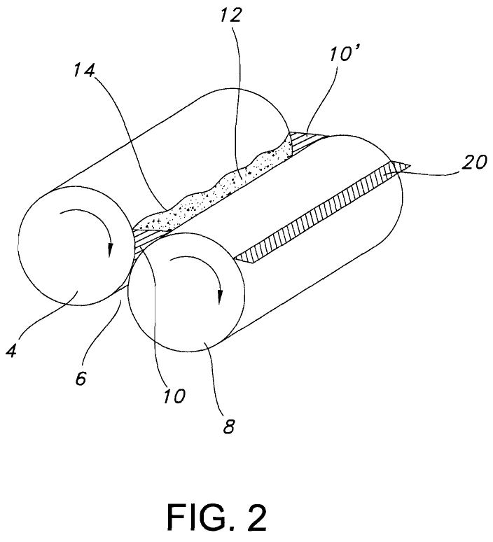

As explained above, the generally V-shaped transfer trough 14 is formed by the space created between the drive roll 4 and the doctor roll 8. Preferably the drive roll 4 and the doctor roll 8 are not located in the same horizontal plane, and most preferably the doctor roll 8 is located in a lower plane than the drive roll 4. As can be seen m FIG. 2, the level of the transfer trough 14 has a “maximum transfer trough volume”, which is equal to the top of the doctor roll 8. Preferably, the level of suspension 12 in the transfer trough 14 is high, above 50% of the maximum transfer trough volume, and most preferably is at least 80% of the maximum transfer trough volume. As used herein, a “high transfer trough volume” means a transfer trough 14 that has enough coating suspension 12 to fill at least 50% of the maximum transfer trough volume. Accordingly, to maintain a high transfer trough volume, the coating suspension 12 should be fed into the transfer trough 14 at a rate that is sufficient to account for the loss of coating suspension 12 as it is adhered to the substrate 2. The level of the suspension 12 in the transfer trough 14 is controlled, as is the rate that the composition 12 is directed into the transfer trough 14. Maintaining the rate and level helps maintain the uniformity of the suspension 12 in the transfer trough 14, and in trim the uniformity in the film sheet. Doctor roll 8 optionally includes a doctor blade 20, which helps scrape the outside of the doctor roll 8, keeping excess coating suspension 12 in the transfer trough 14.

The coating suspension 12 is then coated onto a sheet or film 2. Preferably, the film is prepared by roll coating the film onto a substrate 2, such as paper or Mylar. The substrate 2 is fed along the side of the drive roll 4, passing through the transfer trough 14 and through the gap 6 created by the separation between the drive roll 4 and the doctor roll 8. As the substrate 2 passes through the transfer trough 14, an amount of coating suspension 12 is adhered to the substrate 2. The coated substrate then passes through the gap 6, and enters a drying oven. The sheet of film can then be dried as described herein and cut into individually sized pieces of film. The dried film preferably has a thickness of from about 3 μm to about 250 μm, or about 0.1 mils to about 10 mils. The thickness of the film can be varied depending on the size of the gap 6, and the doctor roll 8 may be moved accordingly. Desirably, the dried film will have a thickness of about 2 mils to about 8 mils, and more desirably, from about 3 mils to about 6 mils. Preferably, the ratio of solids to liquids in the film is at least about 75 to about 25, and more preferably about 95 to about 5, and most preferably about 99 to about 1.

If a multi-layered film is desired, this may be accomplished by coating, spreading, or casting a combination onto an already formed film layer.

Although a variety of different film-forming techniques may be used, it may be desirable to select a method that will provide a flexible film, such as reverse roll coating. The flexibility of the film allows for the sheets of film to be rolled and transported for storage or prior to being cut into individual dosage forms. Desirably, the films will also be self-supporting or in other words able to maintain their integrity and structure in the absence of a separate support. Furthermore, the films of the present invention may be selected of materials that are edible or ingestible.

Coating or casting methods are particularly useful for the purpose of forming the films of the present invention. Specific examples include reverse roll coating, gravure coating, immersion or dip coating, metering rod or meyer bar coating, slot die or extrusion coating, gap or knife over roll coating, air knife coating, curtain coating, or combinations thereof, especially when a multi-layered film is desired.

Roll coating, or more specifically reverse roll coating, is particularly desired when forming films in accordance with the present invention. This procedure provides excellent control and uniformity of the resulting films, which is desired in the present invention. In this procedure, the coating material is measured onto the applicator roller by the precision setting of the gap between the upper metering roller and the application roller below it. The coating is transferred from the application roller to the substrate as it passes around the support roller adjacent to the application roller. Multiple roll processes, such as three roll and four roll processes, are commonly known in the art.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller.

Offset Gravure is common, where the coating is deposited on an intermediate roller before transfer to the substrate.

In the process of immersion or dip coating, the substrate is dipped into a bath of the coating, which is normally of a low viscosity to enable the coating to run back into the bath as the substrate emerges.

In the metering rod coating process, an excess of the coating is deposited onto the substrate as it passes over the bath roller. The wire-wound metering rod, sometimes known as a Meyer Bar, allows the desired quantity of the coating to remain on the substrate. The quantity is determined by the diameter of the wire used on the rod.

In the slot die process, the coating is squeezed out by gravity or under pressure through a slot and onto the substrate. If the coating is 100% solids, the process is termned “extrusion” and in this case, the line speed is frequently much faster than the speed of the extrusion. This enables coatings to be considerably thinner than the width of the slot.

The gap or knife over roll process relies on a coating being applied to the substrate which then passes through a “gap” between a “knife” and a support roller. As the coating and substrate pass through, the excess is scraped off.

Air knife coating is where the coating is applied to the substrate and the excess is “blown off” by a powerful jet from the air knife. This procedure is useful for aqueous coatings.

In the curtain coating process, a bath with a slot in the base allows a continuous curtain of the coating to fall into the gap between two conveyors. The object to be coated is passed along the conveyor at a controlled speed and so receives the coating on its upper face;

### Drying the Film

A controlled drying process may be used to dry the film, and may additionally reduce the variability of the composition in the film. An alternative method of forming a film with an accurate dosage, that would not necessitate the controlled drying process, would be to cast the films on a predetermined well. With this method, although the components may aggregate, this will not result in the migration of the active to an adjacent dosage form, since each well may define the dosage unit per se. When a controlled or rapid drying process is desired, this may be through a variety of methods. A variety of methods may be used including those that require the application of heat. The liquid carriers are removed from the film in a manner such that the uniformity, or more specifically, the non-self-aggregating uniform heterogeneity, that is obtained in the wet film is maintained.

Desirably, the film is dried from the bottom of the film to the top of the film. More desirably, substantially no air flow is present across the top of the film during its initial setting period, during which a solid, visco-elastic structure is formed. This can take place within the first few minutes, e.g. about the first 0.5 to about 4.0 minutes of the drying process. Controlling the drying in this manner prevents the destruction and reformation of the film's top surface. This is accomplished by forming the film and placing it on the top side of a surface having top and bottom sides. Then, heat is initially applied to the bottom side of the film to provide the necessary energy to evaporate or otherwise remove the liquid carrier. The films dried in this manner dry more quickly and evenly as compared to air-dried films, or those dried by conventional drying means. In contrast to an air-dried film that dries first at the top and edges, the films dried by applying heat to the bottom dry simultaneously at the center as well as at the edges. This also prevents settling of ingredients that occurs with films dried by conventional means.

The temperature at which the films are dried is about 100° C. or less, desirably about 90° C. or less, and most desirably about 80° C. or less.

Another method of controlling the drying process, which may be used alone or in combination with other controlled methods as disclosed above includes controlling and modifying the humidity within the drying apparatus where the film is being dried. In this manner, the premature drying of the top surface of the film is avoided.

Additionally, the length of drying time can be properly controlled, i.e. balanced, with the heat sensitivity and volatility of the components, and particularly the flavor oils and drugs. The amount of energy, temperature and length and speed of the conveyor can be balanced to accommodate such actives and to minimize loss, degradation or ineffectiveness in the final film.

The films may initially have a thickness of about 500 μm to about 1,560, μm, or about 20 mils to about 60 mils, which may be controlled by varying the gap between the doctor roll and drive roll, and when dried have a thickness in the desired range set forth above.

### Uses of Thin Films

The thin films of the present invention are well suited for many uses. The high degree of uniformity of the components of the film makes them particularly well suited for incorporating pharmaceuticals. Furthermore, the polymers used in construction of the films may be chosen to allow for a range of disintegration times for the films. A variation or extension in the time over which a film will disintegrate may achieve control over the rate that the active is released, which may allow for a sustained release delivery system. In addition, the films may be used for the administration of an active to any of several body surfaces, especially those including mucous membranes, such as oral, anal, vaginal, ophthalmological, the surface of a wound, either on a skin surface or within a body such as during surgery, and similar surfaces.

The films may be used to orally administer an active. This is accomplished by preparing the films as described above and introducing them to the oral cavity of a mammal. This film may be prepared and adhered to a second or support layer from which it is removed prior to use, i.e. introduction to the oral cavity. An adhesive may be used to attach the film to the support or backing material which may be any of those known in the art, and is preferably not water soluble. If an adhesive is used, it will desirably be a food grade adhesive that is ingestible and does not alter the properties of the active. Mucoadhesive compositions are particularly useful. The film compositions in many cases serve as mucoadhesives themselves.

The films may be applied under or to the tongue of the mammal. When this is desired, a specific film shape, corresponding to the shape of the tongue may be preferred. Therefore the film may be cut to a shape where the side of the film corresponding to the back of the tongue will be longer than the side corresponding to the front of the tongue. Specifically, the desired shape may be that of a triangle or trapezoid. Desirably, the film will adhere to the oral cavity preventing it from being ejected from the oral cavity and permitting more of the active to be introduced to the oral cavity as the film dissolves.

Another use for the films of the present invention takes advantage of the films' tendency to dissolve quickly when introduce to a liquid. An active may be introduced to a liquid by preparing a film in accordance with the present invention, introducing it to a liquid, and allowing it to dissolve. This may be used either to prepare a liquid dosage form of an active, or to flavor a beverage.

The films of the present invention are desirably packaged in sealed, air and moisture resistant packages to protect the active from exposure oxidation, hydrolysis, volatilization and interaction with the environment. Additionally, they can include a dispenser, which may contain a full supply of the medication typically prescribed for the intended therapy, but due to the thinness of the film and package, is smaller and more convenient than traditional bottles used for tablets, capsules and liquids. Moreover, the films of the present invention dissolve quickly upon contact with saliva or mucosal membrane areas, eliminating the need to wash the dose down with water.

Desirably, a series of such unit doses are packaged together in accordance with the prescribed regimen or treatment, e.g., a 10-90 day supply, depending on the particular therapy. The individual films can be packaged on a backing and peeled off for use.

The features and advantages of the present invention are more fully shown by the following examples which are provided for purposes of illustration, and are not to be construed as limiting the invention in any way.

### EXAMPLES

While there have been described what are presently believed to be the preferred embodiments of the invention, those skilled in the art will realize that changes and modifications may be made thereto without departing from the spirit of the invention, and it is intended to include all such changes and modifications as fall within the true scope of the invention. Two sets of experiments were conducted to study the effect of variables on the uniformity of dextromethorphan 7.5 mg strip products.

Experiment Set I

The first Experiment Set attempted to investigate the effects of several specific parameters, including the following: (1) the effect of the doctor roll being in the ON vs. the OFF position; (2) investigation of the effect of using coated API particles, that were sieved to eliminate larger particles and fines from the bulk particles; (3) the effect of sieved particles and the doctor roll being in the ON vs. the OFF position; (4) the effect of sieved particles, the doctor roll being in the ON and OFF positions, and the effect of varying levels of the suspension within the transfer trough; (5) the effect of increasing coat weight with the doctor roll in the ON vs. the OFF position; and (6) the effect of reduction of the percentage of particles in solution.

For this experiment set, the formulation of the 7.5 mg dextromethorphan and the 15 mg dextromethorphan samples used are as set forth in Table 1.

TABLE 1

| | 15 mg | 7.5 mg |
|---|---|---|
| Description | dextromethorphan | dextromethorphan |
| Dextromethorphan HBr | 31.25% | 25.00% |
| Polyethylene oxide | 22.76% | 26.79% |
| Polydextrose | 15.34% | 18.05% |
| Methocel | 11.38% | 13.39% |
| Vicks Cough Drop Cherry | 10.00% | 7.00% |
| Sucralose | 2.05% | 2.50% |
| Magnesium Stearate | 1.82% | 1.82% |
| Sodium Bicarbonate | 1.00% | 1.00% |
| Cool Key | 1.00% | 1.00% |
| Menthol | 1.00% | 1.00% |
| Xantural | 0.75% | 0.75% |
| Titanium dioxide | 0.50% | 1.00% |
| Magna Sweet | 0.50% | 0.50% |
| Color(s) | 0.10% | 0.10% |
| Butylated hydroxyl toluene, | 0.10% | 0.10% |
| NF | | |

Control Experiment

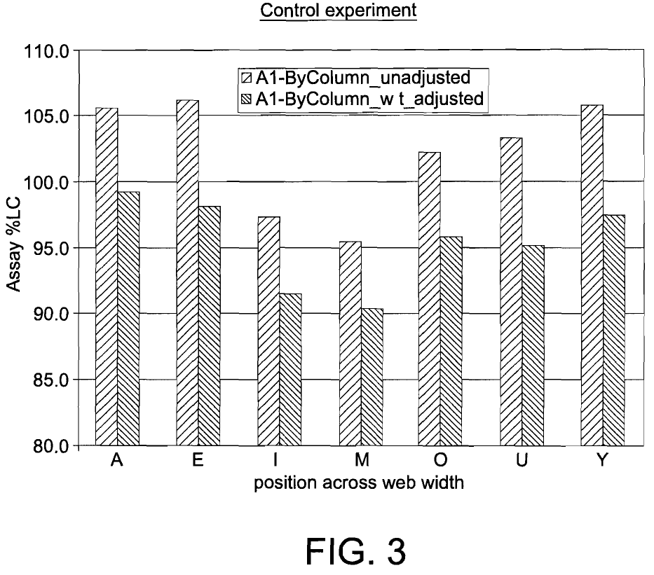

Using a control experiment, it was seen that there was a significant “hammock effect” across the web of the film. The edges of the web were much more concentrated than were the middle portions of the web. This effect is demonstrated in FIG. 3.

Effect of Doctor Roll Turned On

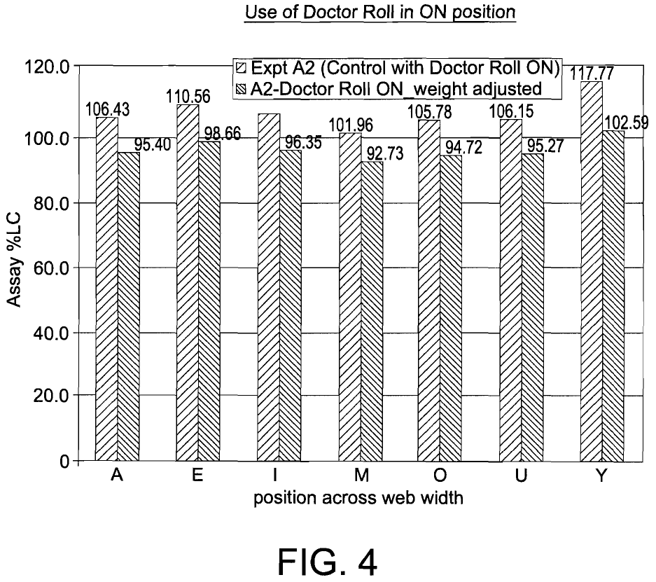

The effect of using the doctor roll in the ON position was effective, but still created some variability throughout the web, especially towards the edge. This effect can be seen in FIG. 4.

Use of Sieved Particles With Doctor Roll On

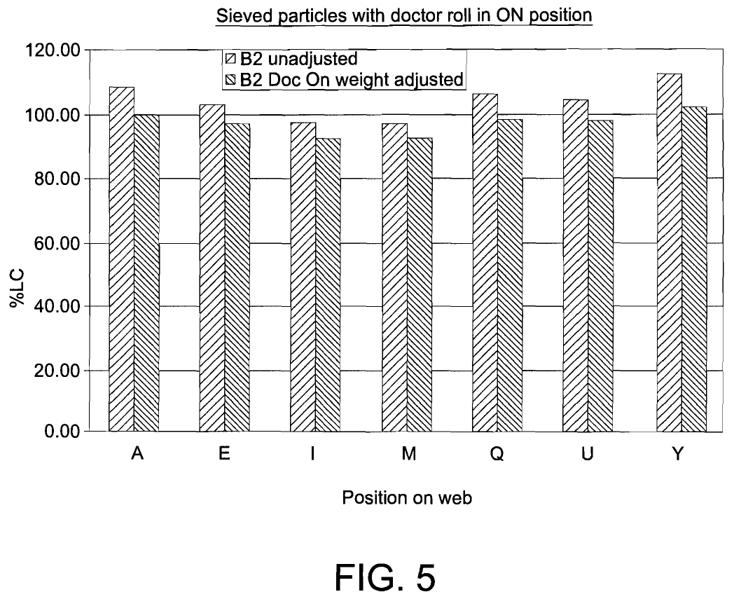

Next the effect of using uniformly-sized particles was examined. The coated dextromethorphan particles were sieved to remove large particle size fraction greater than 70 mesh, and fines smaller than 140 mesh. The effect of using sieved particles resulted in a more uniform distribution of particles throughout the web. The effect of using sieved particles in conjunction with the doctor roll being in the ON position significantly reduced the variability, throughout the web. FIG. 5 shows the effect of using sieved particles with the doctor roll in the ON position.

Use of High Level of Suspension in the Transfer Trough

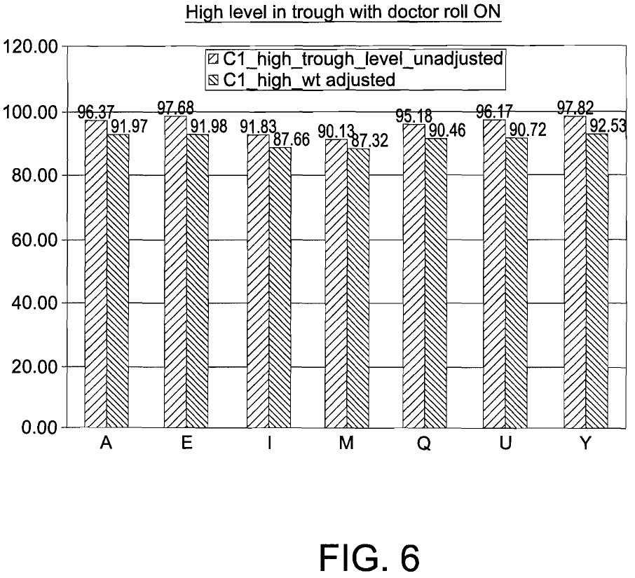

Next, the effect of using a high level of suspension in the transfer trough was examined. While there was some degree of uniformity with the doctor roll turned in the OFF position, the high transfer trough level with the doctor roll in the ON position gave a significantly more uniform distribution throughout the web. FIG. 6 shows the effect of the high transfer trough level with the doctor roll in the ON position.

Effect of Coat Weight on Particles

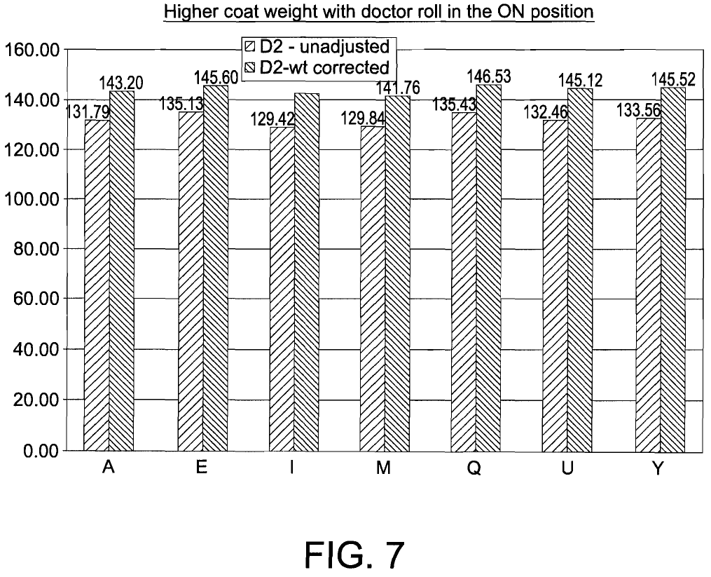

Next, the effect of the coat weight on the particles was examined. The coat weight on the 7.5 mg dextromethorphan particles was increased to about 72 mg/in². Again, the higher coat weight reduced variability throughout the web when the doctor roll was turned ON. The effect of the high coat weight with the doctor roll in the ON position is shown in FIG. 7.

Effect of Using Varying Concentrations of Solids

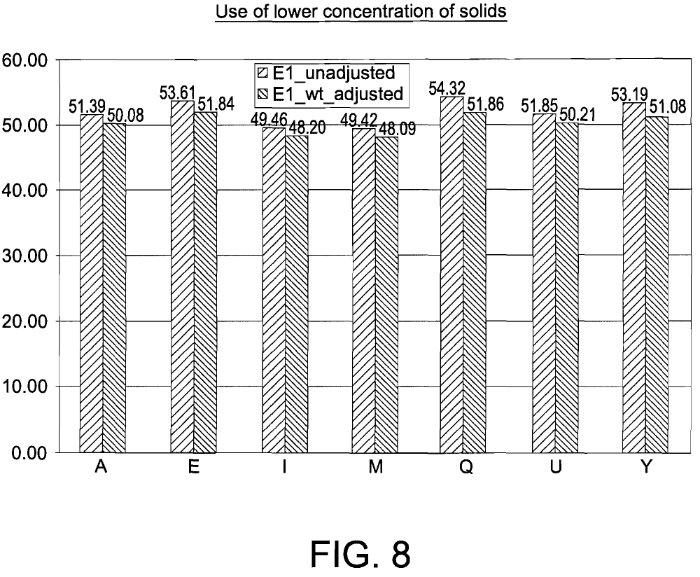

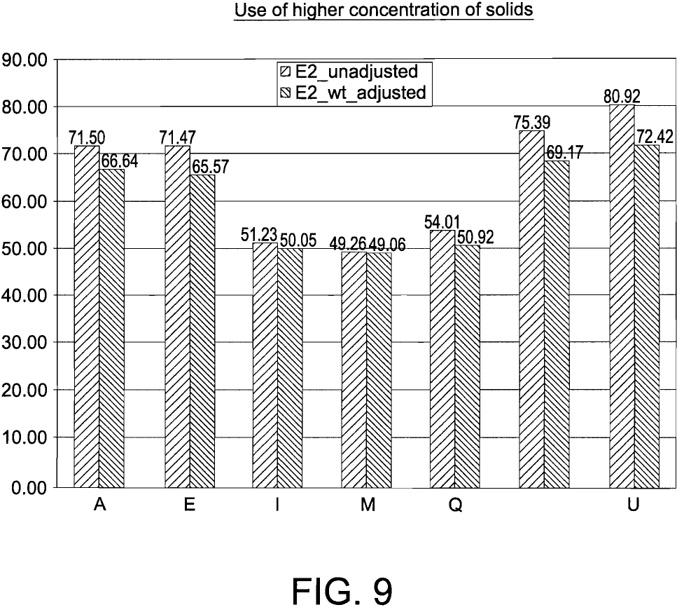

Finally, the effect of using a lower concentration of solids in the suspension was examined. The percentage of solids was reduced to approximately the level that they would be in a formulation containing approximately 3-4 mg per strip. The effect of lower concentration of solids created a more uniform distribution throughout the web than did a higher concentration of solids. This effect can be seen in FIGS. 8 and 9.

Experiment Set II

A second set of experiments was carried out to further investigate certain effects of various processing parameters. Specifically, the experiments were as follows: (1) the effect of turning the doctor roll ON at various speeds of 30%, 60% and 100%. of the maximum speed; (2) the effect of the suspension hose entry point, with the suspension being entered either on the drive side or the operator side; (3) the effect of manually ladling the suspension with a spatula; (4) the effect of using a manifold to spread out the suspension entry over the entire length of the transfer trough instead of at one single point; (5) the effect of using a mylar substrate instead of paper; (6) and the effect of using conditions that were expected to perform best in terms of minimizing variability across the web, i.e., using sieved particles, a high transfer trough level, and a manifold splitting the suspension into streams of entry.

Control Experiment

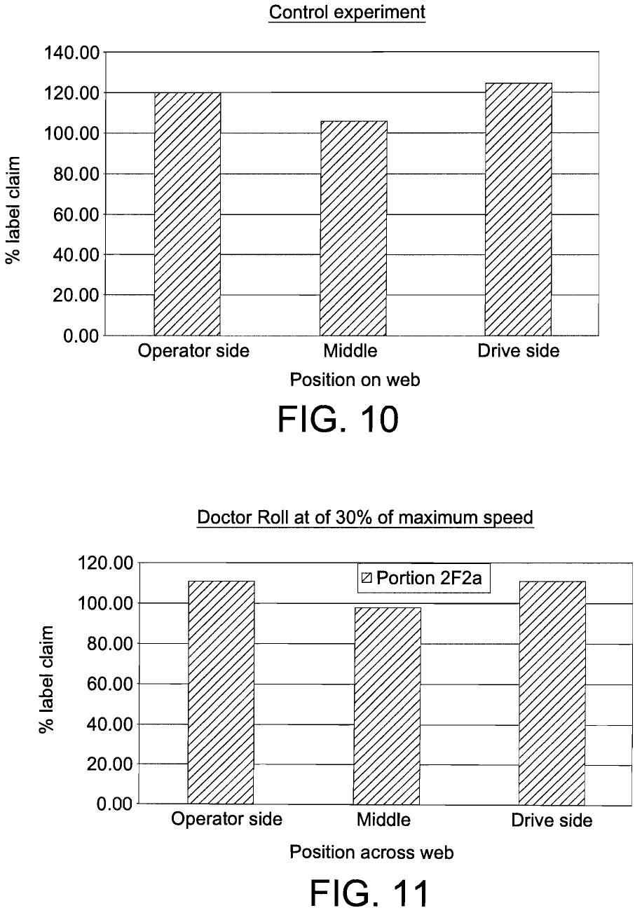

Again, a control set was created, with unsieved particles, the suspension entry hose at mid point, and doctor roll turned OFF. The result was the “hammock effect” described herein, wherein there is a higher concentration of active ingredients at the edges of the web than in the middle of the web. The control experiment showing the “hammock effect” can be seen in FIG. 10.

Effect of Varying Speeds of Doctor Roll

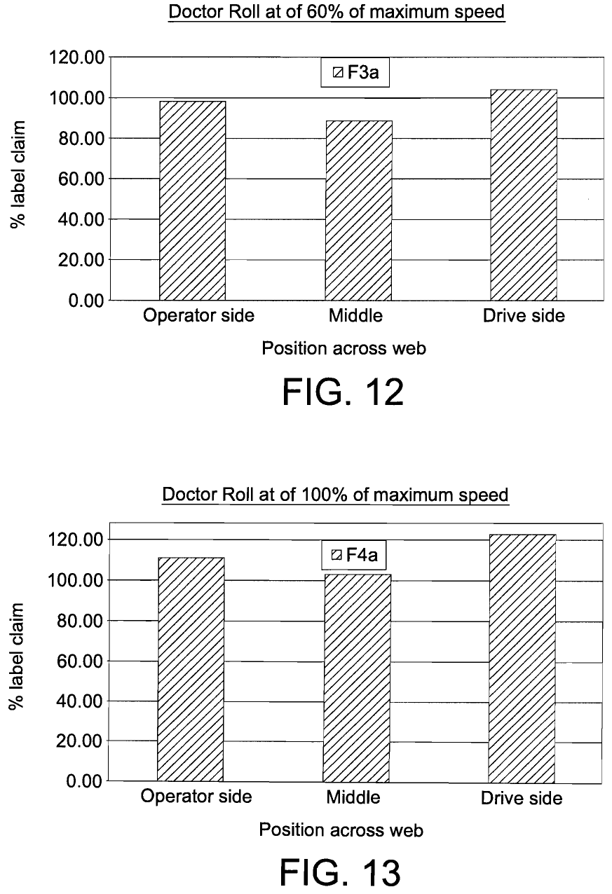

The effect of varying speeds of the doctor roll was then examined. The doctor roll was tested at speeds of 30%, 60% and 100% of maximurrm speed. As can be seen in FIGS. 11-13, there was still a significant “hammock effect” at each of the speeds tested.

Effect of Moving Suspension Point

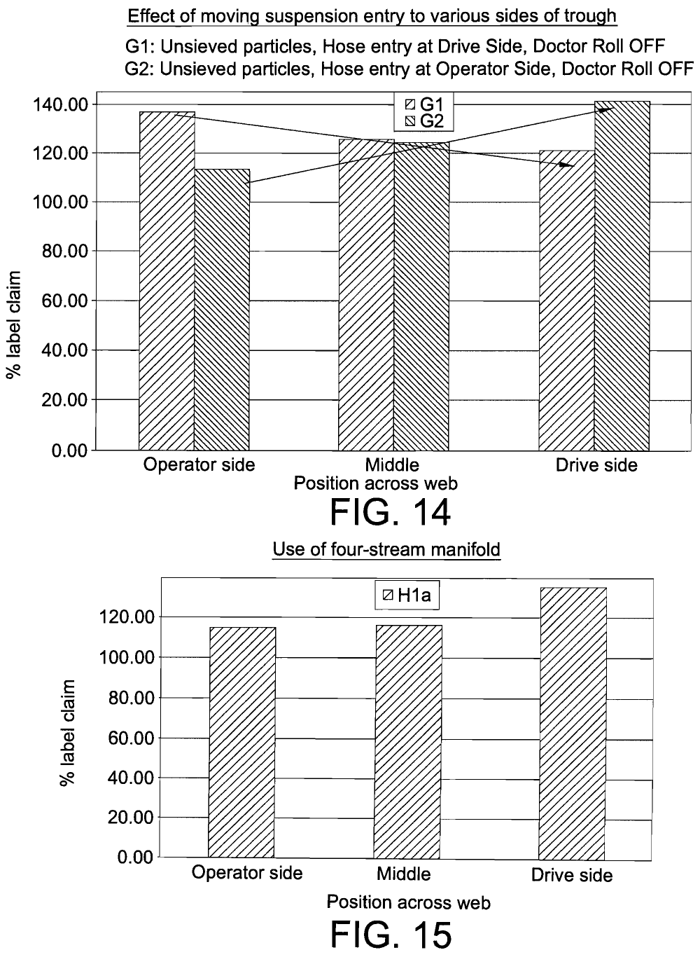

The effect of moving the suspension entry hose from one side of the transfer trough to the other side was examined. A “skewed hammock” resulted, wherein there are higher degrees of materials at the end across the hose entry point. When the hose entry was at the drive side, the operator side contained a higher percentage of materials. When the hose entry was at the operator side, a higher percentage of materials accumulated at the drive side. These results are shown in FIG. 14.

Effect of Using a Four-Stream Manifold

The next examination was the effect of using a specially constructed manifold to split the incoming stream of coating suspension into four separate streams into the transfer trough. The streams each entered the transfer trough a few inches from each other. As can be seen in FIG. 15, the drive side assay value was higher, but the mid-point and the operator side were approximately equal.

Effect of Ladling Suspension into Transfer Trough

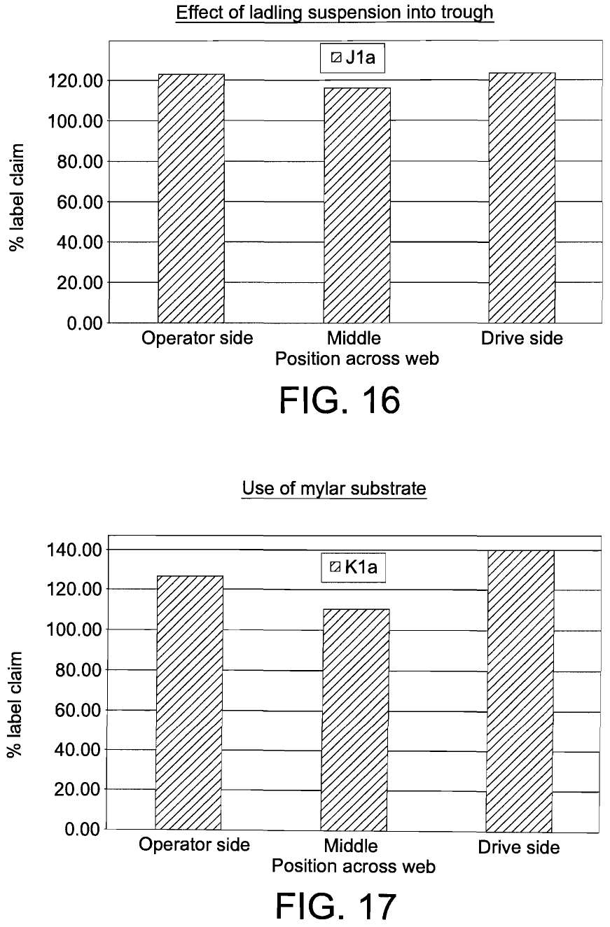

The effect of ladling the suspension into the transfer trough was then examined. The suspension was manually moved around with a rubber spatula. As can be seen in FIG. 16, the variability was reduced.

Effect of Using Mylar Substrate

The effect of using a mylar substrate instead of a paper substrate was the examined. The experiment resulted in the same “hammock effect” seen in the control test. This is shown in FIG. 17.

Effect of Using Sieved Particles, High Trough Level, and Split Stream

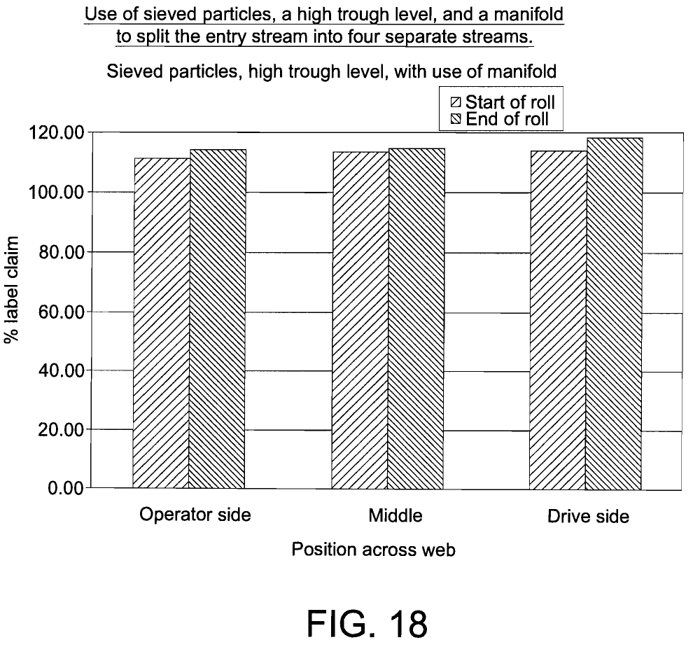

Finally, the effect of using sieved particles, a high trough level, and a split stream manifold was examined As can be seen in FIG. 18, this resulted in a nearly uniform distribution of particles throughout the web at both the start of the roll and the end of the roll.

It should be understood that the foregoing examples are for reference only, and are not intended to limit the scope of the present invention in any way.

What is claimed is:

1. A method of making a film product comprising the steps of:
a. forming a flowable film composition comprising an edible water-soluble polymer, a polar solvent and a particulate active suspended therein, wherein the ratio of solids to liquids is at least about 95 to about 5 and wherein the composition comprises a mixture having compositional uniformity per unit volume;
b. controllably directing said flowable film composition to a transfer trough, said transfer trough comprising a first rotating roller and a movable transfer substrate upon which to form the film; and
c. maintaining the rate of composition entering the transfer trough and the level of the flowable film composition in the transfer trough at a substantially constant level.

2. The method of claim 1, further comprising the step of using multiple delivery conduits to controllably direct said flowable film composition to said transfer trough.

3. The method of claim 2, wherein said multiple delivery conduits are positioned along the length of the transfer trough to reduce the flow currents from disturbing the uniformity in the transfer trough.

4. The method of claim 1 wherein the transfer trough has a volume of at least about 50% of the maximum transfer trough volume.

5. The method of claim 1 wherein said particulate active has an average particle size of about 100 micrometers to about 200 micrometers.

6. The method of claim 1 wherein said polar solvent includes water.

7. The method of claim 1 wherein said water-soluble polymer includes a cellulose derivative.

8. The method of claim 1, wherein said step of directing said flowable film composition to a transfer trough is performed via using a ladle to manually feed an amount of the flowable film composition into said transfer trough.

9. A film product prepared by the method of claim 1.

10. A method of orally administering an active to an individual comprising the steps of:
a. preparing a film by the steps of:
a. forming a flowable film composition comprising an edible water-soluble polymer, a polar solvent and a particulate active suspended therein, wherein the ratio of solids to liquids is at least about 95 to about 5 and wherein the composition comprises a mixture having compositional uniformity per unit volume;
b. controllably directing said flowable film composition to a transfer trough, said transfer trough comprising a first rotating roller and a movable transfer substrate upon which to form the film; and
c. maintaining the rate of composition entering the transfer trough and the level of the flowable film composition in the transfer trough at a substantially constant level; and
b. introducing said film to the oral cavity of an individual.

11. The method of claim 10, further comprising the step of using multiple delivery conduits to controllably direct said flowable film composition to said transfer trough.

12. The method of claim 11, wherein said multiple delivery conduits are positioned along the length of the transfer trough to reduce the flow currents from disturbing the uniformity in the transfer trough.

13. The method of claim 10 wherein the transfer trough has a volume of at least about 50% of the maximum transfer trough volume.

14. The method of claim 10 wherein said particulate active has an average particle size of about 100 micrometers to about 200 micrometers.

15. The method of claim 10 wherein said polar solvent includes water.

16. The method of claim 10 wherein said water-soluble polymer includes a cellulose derivative.

17. The method of claim 10, wherein said step of directing said flowable film composition to a transfer trough is performed via using a ladle to manually feed an amount of the flowable film composition into said transfer trough.

18. A method of introducing an active component to a liquid comprising the steps of:
a. preparing a film by the steps of:
i. forming a flowable film composition comprising an edible water-soluble polymer, a polar solvent and a particulate active suspended therein, wherein the ratio of solids to liquids is at least about 95 to about 5 and wherein the composition comprises a mixture having compositional uniformity per unit volume;
ii. controllably directing said flowable film composition to a transfer trough, said transfer trough comprising a first rotating roller and a movable transfer substrate upon which to form the film; and
iii. maintaining the rate of composition entering the transfer trough and the level of the flowable film composition in the transfer trough at a substantially constant level;
b. placing said film into a liquid; and
c. allowing said film to dissolve.

19. The method of claim 18, further comprising the step of using multiple delivery conduits to controllably direct said flowable film composition to said transfer trough.

20. The method of claim 19, wherein said multiple delivery conduits are positioned along the length of the transfer trough to reduce the flow currents from disturbing the uniformity in the transfer trough.

21. The method of claim 18 wherein the transfer trough has a volume of at least about 50% of the maximum transfer trough volume.

22. The method of claim 18 wherein said particulate active has an average particle size of about 100 micrometers to about 200 micrometers.

23. The method of claim 18 wherein said polar solvent includes water.

24. The method of claim 18 wherein said water-soluble polymer includes a cellulose derivative.

25. The method of claim 18, wherein said step of directing said flowable film composition to a transfer trough is performed via using a ladle to manually feed an amount of the flowable film composition into said transfer trough.
